(19) United States
(12) Patent Application Publication
(10) Pub. No.: US 2013/0116413 A1
(43) Pub. Date: May 9, 2013

(54) PURIFICATION OF PROTEINS

(71) Applicants: Samir Kulkarni, Chandivli (IN); Satyam Subrahmanyam, Masab Tank (IN); Y. Raghvendra, Kesevayanagunta (IN); Prashant Kardekar, Geetanjali (IN)

(72) Inventors: Samir Kulkarni, Chandivli (IN); Satyam Subrahmanyam, Masab Tank (IN); Y. Raghvendra, Kesevayanagunta (IN); Prashant Kardekar, Geetanjali (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/518,532

(22) PCT Filed: Dec. 28, 2010
(86) PCT No.: PCT/US10/62246

§ 371 Date: Sep. 19, 2012

(30) Foreign Application Priority Data
Dec. 29, 2009 (IN) 3214/CHE/2009

(51) Int. Cl. C07K 1/36

(52) U.S. Cl. CPC C07K 1/36
USPC 530/387.1

(57) ABSTRACT

The invention describes a method for protein purification. More particularly, the invention relates to a purification process comprising protein A chromatography and anion exchange chromatography wherein protein A chromatography eluate is further purified by anion exchange chromatography at similar pH or at a pH less than or equal to 6.

Aspects of this application relate to purification of proteins using chromatographic methods. In embodiments, purification is conducted at low pH values. In particular embodiments, the application relates to purification processes comprising protein A chromatography and anion exchange chromatography, wherein a protein A chromatography eluate is further purified by anion exchange chromatography at similar pH values, or at pH values less than or equal to 6.

Large scale purification of proteins remains a significant challenge in the biopharmaceutical industry, as efficient and cost-effective methods are required to achieve desired yields and purity levels. Therapeutic proteins are primarily produced by recombinant DNA technology, i.e., by cloning and expression of a heterologous gene in prokaryotic or eukaryotic systems. However, proteins expressed by recombinant DNA methods are typically associated with contaminants such as host cell proteins (“HCP”), host cell DNA (“HCD”), viruses, etc. The presence of these contaminants is a potential health risk, and hence their removal from final products is a regulatory requirement. Thus, drug regulatory agencies such as United States Food and Drug Administration (“FDA”) require that biopharmaceuticals be free from impurities, both product related (aggregates or degradation products) and process related (media components, HCP, DNA, chromatographic media used in purification, endotoxins, viruses, etc.). See FDA Office of Biologics Research and Review, Points to consider in the production and testing of new drugs and biologicals produced by recombinant DNA technology (Draft), 1985. Thus, elimination of impurities and contaminants from final products is mandatory and poses a significant challenge in the development of methods for the purification of proteins.

Protein purification is frequently a multistep process, wherein different chromatographic steps are performed sequentially to yield a final purified product. For purification of monoclonal antibodies, protein A chromatography is one of the widely used methods and can be the first step in antibody purification. This is a type of affinity chromatography, wherein separation is affected by means of a resin tagged with protein A (Hjelm H. et.al., FEBS lett. 1972; 28, 73-76; Langone J J., Adv Immunol, 1982; 32, 157-252). The various aspects of Protein A chromatography (protein A and its variants, chromatographic medium, etc.) have been described in U.S. Pat. Nos. 6,013,763 and 6,399,750, and European Patent Application Publication Nos. 282308 and 284368. A disadvantage of protein A chromatography is the leaching of Protein A and its fragments from the chromatographic resin and its contamination of the eluate. Since protein A is of bacterial origin (obtained from Staphylococcus aureus), it's removal is necessary to avoid undesirable immune responses. Blaint et. al. have shown that IgG can form complexes with protein A that may activate Fc bearing leukocytes and complement system to generate oxidant and anaphylatoxin activity in vitro (Balint J. et.al., Cancer Res. 1984; 44, 734-743). Further, protein A has also been linked with toxicity (Bensinger W I. et. al., J. Biol Resp. Modif. 1984; 3, 347; Messeschimdt G L. et. al., J. Biol. Resp. Modif. 1984; 3, 325; Terman D. S. and Bertram, J. H., Eur. J. Cancer Clin. Oncol. 1985; 21, 1115 and Ventura G. J et. al., Cancer Treat. Rep. 1987; 71, 411). Thus subsequent purification steps are required to remove protein A leachates, as well as residual host cell proteins, host cell DNA, etc., to meet regulatory requirements.

The literature discloses various methods for purification of crude or partially purified samples. Balint et al. describe the use of gel filtration for separating uncomplexed antibodies from IgG-protein A complexes (Balint et.al., Cancer Res 1984; 44, 734-743). U.S. Pat. No. 4,983,722, European Patent Application Publication No. 1601697, and U.S. Patent Application Publication No. 2007/0292442 describe the use of ion exchange chromatography for purification of antibodies. However, these methods either result in considerable losses of antibody or require substantial pH adjustment of the sample prior to a chromatography step. The change in pH is achieved by addition of a high molarity base that compromises process efficiency as a result of volume dilution and mixing efficiency, as well as product stability due to localized pH surge. The impact on product stability is of particular significance as it leads to significant product loss due to denaturation, precipitation and aggregation.

Improved processes for purifying proteins are needed.

### SUMMARY

In aspects, the application describes purification methods comprising multiple chromatographic steps, wherein in embodiments a low pH eluate from protein A chromatography is further purified by anion exchange chromatography at about the same pH, or at pH values less than or equal to 6.

### BRIEF DESCRIPTION OF THE DRAWINGS

FIG. 1 is an illustration of a chromatogram from the procedure of Example 1.

FIG. 2 is an illustration of a chromatogram from the procedure of Example 2.

FIG. 3 is an illustration of a chromatogram from the procedure of Example 2.

FIG. 4 is an illustration of a chromatogram from the procedure of Example 3.

FIG. 5 is an illustration of a chromatogram from the procedure of Example 3.

### DETAILED DESCRIPTION

The term “flow-through mode” as used herein refers to chromatographic methods wherein a desired protein is obtained in the flow-through liquid during loading or post load washing of a chromatography column. The desired protein in the flow-through may be collected as various fractions and pooled together or can be collected as a single fraction.

The term “bind-elute mode” as used herein refers to chromatographic methods wherein a desired protein is bound to a chromatography resin when loaded onto a resin column and is subsequently eluted using an elution buffer. The desired protein is collected in elution liquid and may be collected as a single fraction or as various fractions that are pooled together.

The term “antibody” as used herein refers to an immunoglobulin that is composed of four polypeptide chains, consisting of two light and two heavy chains, as well as any immunoglobulins isolated from various sources, such as murine, human, recombinant, etc, truncated antibodies, chimeric, humanized, or pegylated antibodies, isotypes, allotypes, and alleles of immunoglobulin genes. The term antibody as used herein also refers to fusion proteins which contain an immunoglobulin moiety.

The term “low pH” or “acidic pH” as used herein refers to a pH less than or equal to 4.5.

In the purification of monoclonal antibodies (“MAbs”), protein A chromatography is a common method, as highly purified MAbs can be obtained due to the high specificity and binding between protein A ligand and the Fc region of the antibody. However, as discussed earlier, a disadvantage of protein A chromatography is the leaching of protein A and its fragments in the eluate. Hence, further purification steps are required for the removal of protein A and/or its fragments as well as residual host cell proteins, endotoxins, and host cell DNA.

U.S. Pat. No. 4,983,722 and European Patent Application Publication No. 1601697 describe the use of anion exchange chromatography for the purification of proteins. However, the anion exchange step is performed at neutral to alkaline pH values, necessitating pH adjustment of input material. For instance, in U.S. Pat. No. 4,983,722 the protein A eluate is diafiltered against a DEAE equilibration buffer at pH 8.6, while in EP 1601697 the acidic protein A eluate is neutralized with a high molarity buffer such as 0.5 M TrisHCl pH 7.5 and diafiltered with binding/equilibration buffer at pH 8.0, prior to the next chromatographic step. Likewise, U.S. Patent Application Publication No. 2007/0292442 describes the use of two ion exchange resins for the purification of antibodies, wherein the pH of the first eluate is adjusted before loading onto the second ion exchange resin. The pH adjustment greatly compromises both the process efficiency and the product stability. Hence, a process involving no or minimal pH adjustment will be a better alternative to the current methods. The present application describes a process that, in embodiments, virtually eliminates the need of pH adjustment in the purification process, thereby minimizing its impact on process efficiency and product stability.

An aspect of the present application provides methods for antibody purification, embodiments comprising:

1. A first purification step using protein A chromatography, wherein the antibody is eluted at low pH values.

2. A second purification step using anion exchange chromatography that is performed in the flow-through mode, wherein eluate obtained from the first step is loaded onto the anion exchange resin at pH values less than or equal to 6.

In embodiments, the antibody is eluted in the first purification step at pH values about 3.5.

In embodiments, the antibody is loaded onto the anion exchange resin at pH values about 4.

In embodiments, the anion exchange resin is loaded at a pH of 6

An aspect of the present application provides methods for antibody purification, embodiments comprising:

1. A first purification step using protein A chromatography, wherein the antibody is eluted at low pH values.

2. A second purification step using anion exchange chromatography performed in the flow-through mode, wherein eluate obtained from the first step is loaded onto the anion exchange resin without substantial adjustment of pH (viz. within a range of ±0.2 pH values).

In embodiments, the antibody is eluted in the first purification step at pH values about 3.3 to about 4.5 and loaded onto the anion exchange resin at pH values about 3.3 to about 4.5.

In embodiments, the anion exchange chromatography step is followed by a cation exchange chromatography step in a bind-elute mode, wherein the flow-through from the anion exchange chromatography step is loaded onto the cation exchange resin at pH values less than or equal to 6.

The protein A chromatographic resin used may be any protein A or variant or a functional fragment thereof coupled to any chromatographic support. In embodiments, the protein A resin is Prosep vA Ultra® (from Millipore). In embodiments, fresh (i.e., not previously used) protein A chromatographic resin may be used to obtain a feed stream for the second chromatographic step. After washing with loading buffer and intermediate wash, the elution is carried out at low pH values.

Anion exchange chromatography mentioned in the embodiments may be carried out using any weak or strong anion exchange chromatographic resin or a membrane which could function as a weak or a strong anion exchanger. Commercially available anion exchange resins include, but are not limited to, DEAE cellulose, Poros PI 20, PI 50, HQ 10, HQ 20, HQ 50, D 50 from Applied Biosystems, MonoQ, MiniQ, Source 15Q and 30Q, Q, DEAE and ANX Sepharose Fast Flow, Q Sepharose high Performance, QAE SEPHADEX and FAST Q SEPHAROSE from GE Healthcare, Macro-Prep DEAE and Macro-Prep High Q from Biorad, Q-Ceramic Hyper D, DEAE-Ceramic Hyper D, from Pall Corporation. In embodiments of the invention, a strong anion exchange resin, such as Q-Sepharose Fast Flow® (GE Healthcare Life Sciences) is used. This resin is made using a highly cross-linked, 6% agarose matrix attached to —O—CH₂CHOHCH₂OCH₂CHOHCH₂CHOHCH₂N⁺(CH₃)₃ functional group.

Cation exchange chromatographic step mentioned in the embodiments may be carried out using any weak or strong cation exchange chromatographic resin or a membrane which could function as a weak or a strong cation exchanger. Commercially available cation exchange resins include, but are not limited to, those having a sulfonate based group e.g., MonoS, MiniS, Source 15S and 30S, SP Sepharose Fast Flow, SP Sepharose High Performance from GE Healthcare, Toyopearl SP-650S and SP-650M from Tosoh, S-Ceramic Hyper D, from Pall Corporation or a carboxymethyl based group e.g., CM Sepharose Fast Flow from GE Healthcare, Macro-Prep CM from BioRad, CM-Ceramic Hyper D, from Pall Corporation, Toyopearl CM-650S, CM-650M and CM-650C from Tosoh. In embodiments of the invention, a weak cation exchange resin, such as CM Ceramic Hyper D F® (Pall Corporation) is used; this is made using rigid porous beads that are coated with functionalized hydrogel.

Examples of buffering agents used in the buffer solutions include, but are not limited to, TRIS, phosphate, citrate, and acetate salts, or derivatives thereof.

The protein A leachates can be analyzed using protein A ELISA and the purified antibody can be analyzed using protein A high performance liquid chromatography.

Certain specific aspects and embodiments of the application are more fully described by reference to the following examples, being provided only for purposes of illustration. These examples should not be construed as limiting the scope of the application in any manner.

### EXAMPLE 1

### Protein A Chromatography

An anti-VEGF antibody was cloned and expressed in a CHO cell line as described in U.S. Pat. No. 7,060,269, which is incorporated herein by reference. The cell culture broth containing the expressed antibody was harvested, clarified and subjected to protein A affinity chromatography as described below.

The clarified cell culture broth (CCCB) was loaded onto the protein A chromatography column (Prosep vA Ultra, VL44×250, 205 mL) that was pre-equilibrated with 5 column volumes (CV) of equilibration buffer (50 mM Tris, 150 mM NaCl, pH 7.5). The column was then washed with 5 CV of equilibration buffer. This was followed by a wash with 5 CV of 50 mM Tris, 750 mM NaCl, pH 7.5 buffer and a final wash with 25 mM Tris at pH 7.5. The bound antibody was eluted using the low pH buffer 200 mM acetate, pH 3.5.

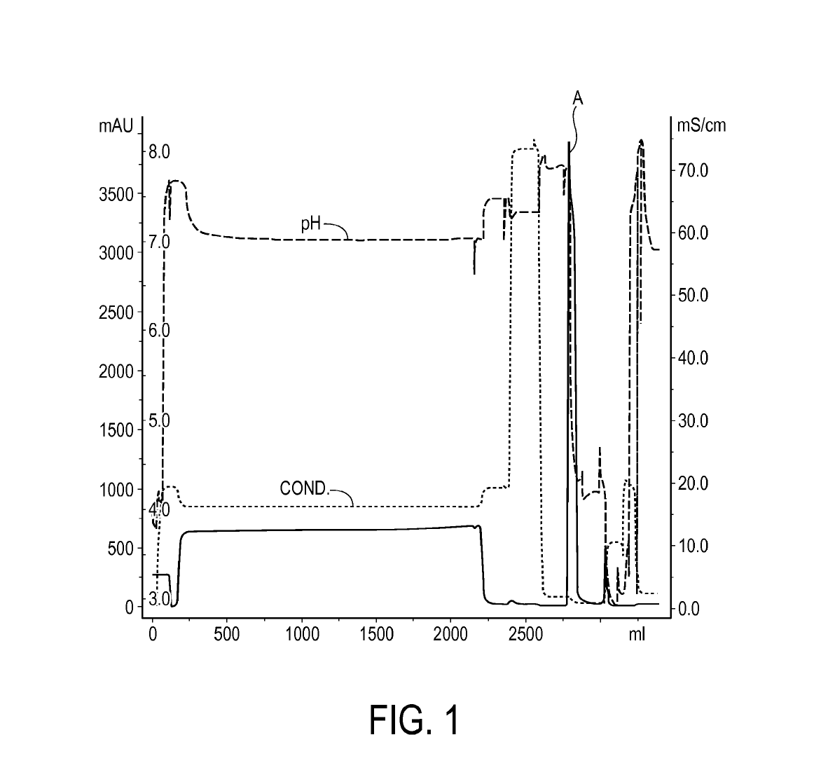

FIG. 1 is an illustration of a chromatogram from the procedure described in this example. The line marked “Cond” represents the increase in conductivity in mS/cm. Peak A represents the eluate obtained from the protein A chromatography resin.

### EXAMPLE 2

### Anion Exchange Chromatography

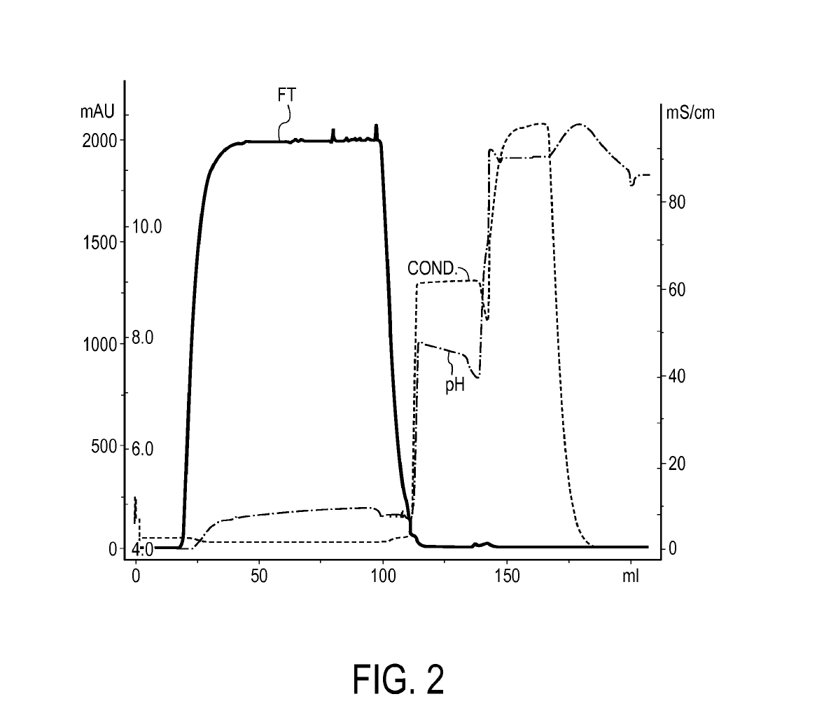

The protein A eluate obtained from Example 1 was incubated at pH 3.5 and 25° C. for 30 minutes for viral inactivation, and the pH was adjusted to 4.0. The sample was then filtered through 0.8/0.2 μm membrane filter and loaded onto an anion exchange resin (Q-Sepharose FF, VL32×250, 80 mL), pre-equilibrated with 5-20 CV of an equilibration buffer (200 mM acetate buffer, pH 4.0). This was followed by a wash with 5 CV of equilibration buffer, and the flow-through during loading and washing steps was collected (corresponding to peak A in FIG. 2) and analyzed for percentage reduction in contaminants or impurities.

Alternatively, protein A eluate may be loaded at pH 3.5, 5.0 or 6.0 onto an anion exchange resin pre-equilibrated with 5-20 CV of equilibration buffer at pH 3.5, 5.0 or 6.0. The resin is washed with 5 CV of equilibration buffer and the load and wash flow-through collected.

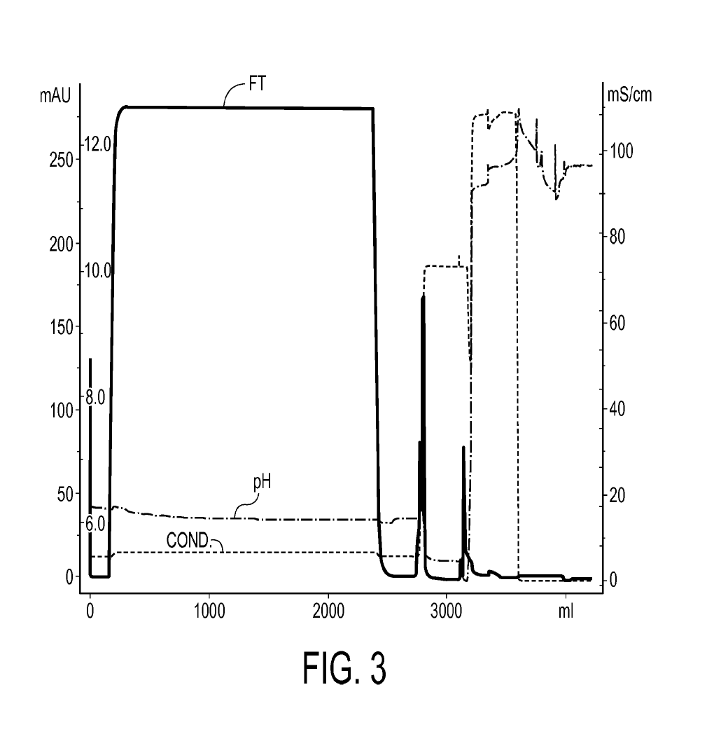

FIGS. 2 and 3 are illustrations of chromatograms from the procedure described in this example, wherein the anion exchange resin is loaded at pH 4.0 and 6.0, respectively. The line marked “Cond” represents the increase in conductivity in mS/cm. “FT” represents the flow-through obtained.

### EXAMPLE 3

### Cation Exchange Chromatography

The flow-through obtained from Example 2 was loaded onto a cation exchange resin (CM Ceramic Hyper D F, VL44×250, 304 mL) pre-equilibrated with 10 CV of equilibration buffer (200 mM acetate buffer, pH 4.0). This was followed by washing with 30 CV of wash buffer (35 mM phosphate buffer pH 6.0). The bound antibody was then eluted using a conductivity gradient (2.5 mS/cm to 7 mS/cm) with a phosphate buffer (35 mM to 80 mM, pH 6.0).

Alternatively the flow-through obtained from Example 2 may be loaded at pH 3.5, 5.0 or 6.0 onto the cation exchange resin pre-equilibrated with 10 CV of equilibration buffer at pH 3.5, 5.0 or 6.0. The resin is washed with 10-30 CV of wash buffer (35 mM Phosphate buffer, pH 6.0). The bound antibody is eluted using a conductivity gradient (2.5 mS/cm to 7 mS/cm) with a phosphate buffer (35 to 80 mM, pH 6.0).

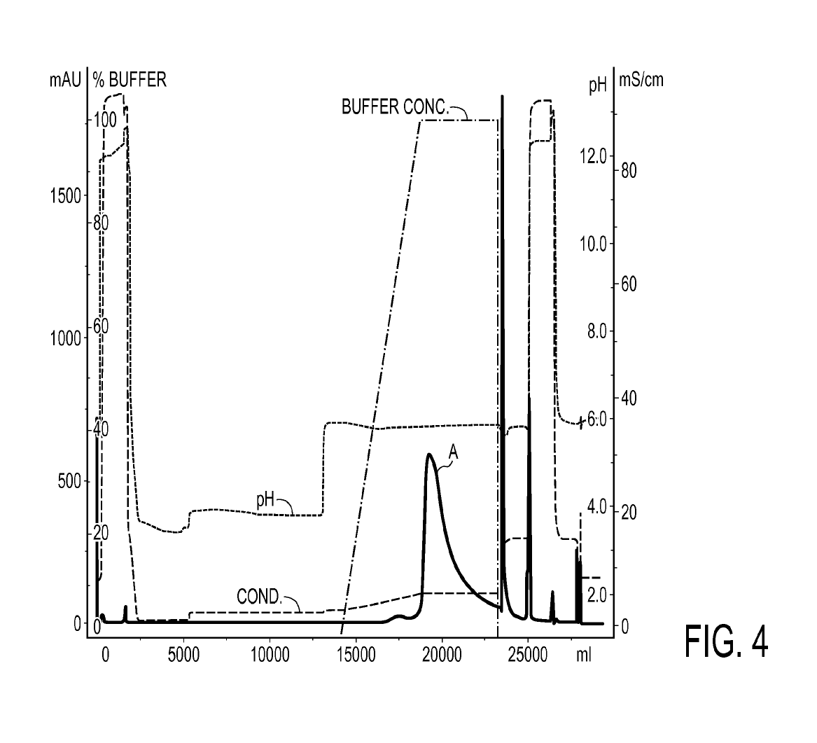

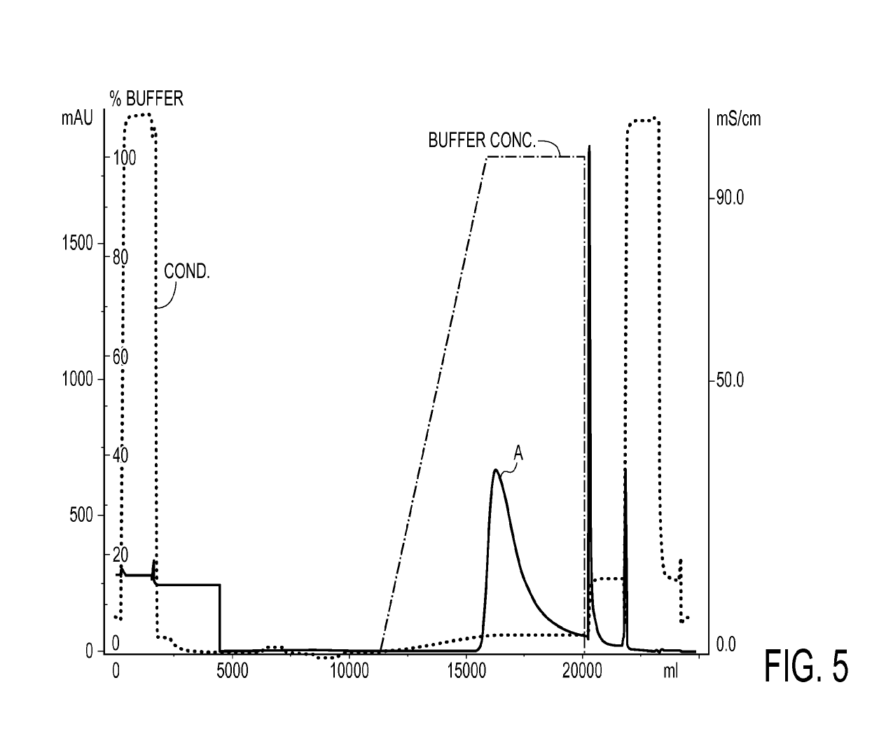

FIGS. 4 and 5 are illustrations of chromatograms from the procedure described in this example, wherein the cation exchange resin is loaded at pH values of 4 and 6, respectively. The line marked “Cond” represents the increase in conductivity in mS/cm. “Buffer Conc.” represents the concentration of phosphate buffer during the chromatography run, where 100% corresponds to a buffer concentration of 80 mM.

Table 1 shows the percentage reductions in the impurities in the anion exchange flow-through and cation exchange eluate.

TABLE 1

| | | % Reduction After Protein A | |
|---|---|---|---|
| | | Chromatography | |
| Purification | | Load Solution | Load Solution |
| Step | Impurity | pH 4 | pH 6 |
| Anion exchange | Protein A leachates | 30 | 80 |
| chromatography | Host cell proteins | 30 | BD |
| Cation exchange | Protein A leachates | BD | BD |
| chromatography | Host cell proteins | BD | BD |

BD: Below detection limit.

1. A process for purifying an antibody, comprising:
a) purifying using protein A chromatography, wherein the antibody is eluted at low pH; and
b) purifying using anion exchange chromatography performed in the flow-through mode, wherein eluate obtained from step a) is loaded onto an anion exchange resin at pH values less than or equal to 6.

2. A process according to claim 1,wherein the antibody is eluted in step a) at pH values about 3.3 to about 4.5 and loaded onto the anion exchange resin at pH values about 3.3 to about 6.

3. A process according to claim 1, wherein the antibody is eluted in step a) at pH values about 3.5 and loaded onto the anion exchange resin at pH values about 3.5 to about 6.

4. A process according to claim 1, wherein the antibody is loaded onto the anion exchange resin at pH values about 4.

5. A process according to claim 1, wherein the antibody is loaded onto the anion exchange resin at pH values about 6.

6. A process according to claim 1, wherein the anion exchange chromatography is followed by a cation exchange chromatography step in a bind-elute mode, and wherein the flow-through from the anion exchange chromatography step is loaded onto a cation exchange resin at pH values less than or equal to 6.

7. A process for purifying an antibody, comprising:
a) purifying using protein A chromatography, wherein the antibody is eluted at low pH values; and
b) purifying using anion exchange chromatography, performed in the flow-through mode, wherein eluate obtained from step a) is loaded on to the anion exchange resin without substantial adjustment of pH.
